(19) United States
(12) Patent Application Publication
(10) Pub. No.: US 2010/0279396 A1
(43) Pub. Date: Nov. 4, 2010

(54) MICRO-SCALED ANIMAL CELL INCUBATOR AND PRODUCTION METHOD THEREOF

(75) Inventors: Yo Han Choi, Daejeon (KR); Kwang Hyo Chung, Daejeon (KR); Dae Sik Lee, Daejeon (KR); Sang Hee Kim, Daejeon (KR); Min Suk Jeong, Jeollabuk-do (KR); Chi Hoon Jun, Daejeon (KR); Moon Youn Jung, Daejeon (KR); Seon Hee Park, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/746,093

(22) PCT Filed: Jun. 27, 2008
(86) PCT No.: PCT/KR2008/003741

§ 371 Date: Jun. 3, 2010

(30) Foreign Application Priority Data
Dec. 6, 2007 (KR) 10-2007-0126314

(51) Int. Cl. C12M 3/00; H05K 3/00

(52) U.S. Cl. 435/294.1; 435/303.1; 29/829

(57) ABSTRACT

There is provided a cell culture technology for culturing an animal cell in a separate microstructure. The micro-scaled animal cell incubator includes a lower glass substrate having fine hot wires processed with a metal and formed in an upper surface thereof; a first PDMS film attached onto the lower glass substrate to form two or more liquid and solid storage spaces in a position corresponding to the fine hot wires of the lower glass substrate and gas flow channels coupled respectively to the liquid and solid storage spaces to allow generated gases to flow therethrough; a gas-permeable PDMS thin film attached onto the gas flow channels of the PDMS film to pass the generated gases therethrough; a second PDMS film attached onto the PDMS thin film and having a culture medium storage space for storing a culture medium; and an upper glass substrate attached onto the second PDMS film and having fine hot wires formed in a lower surface thereof, the fine hot wires being covered by the PDMS film. The micro-scaled animal cell incubator may be useful to control a temperature of a culture medium to a suitable temperature level, as well as to self-supply gases required for the animal cell culture in a microstructure.

### TECHNICAL FIELD

The present invention relates to a cell culture technology for culturing an animal cell in a separate microstructure, and more particularly, to an incubator that self-supplies gases required for the culture of animal cells in a microstructure and also has its own hot wires to control temperature of a culture medium, and a production method thereof.

### BACKGROUND ART

Recently, there has been an increasing demand for a lab-on-a-chip (LOC) technique as one of the miniature analysis systems, and studies on the LOC technique have been widely performed in the art.

Lab-on-a-chip (LOC) is a technique for laminating apparatuses on a chip using micromachining technologies such as photolithography and etching technology, the apparatuses being required for sample analysis (sample pretreatment, reaction, separation and detection apparatuses, etc.), and the chip being formed of glass, plastic or silicon that has a size of several square centimeters (cm²). This is a novel miniature analysis system that is designed to automatically analyze samples in high speed and high efficiency, and one representative example of the miniature analysis system is -total analysis system (TAS).

The LOC technique may be carried out to separate and analyze only a trace of a sample, and therefore has an advantage that this LOC technique may apply to the fields of medical care, diagnosis and biological applications, all of which have difficulty to obtain a large amount of samples. The LOC technique has been widely used for the separation of amino acids and peptides, DNA sequencing, immunoassays, etc., but its applications have been extended into other areas of researches.

In particular, The LOC technique has been increasingly required for the application to the field of separation and analysis of environmental pollutants that should be carried out in real time in job sites, the next-generation filed of analysis and measurements for performing the function of mobile small laboratories that may directly confirm experimental results in job sites, and the drug screening field required for high-speed analysis of many diagnostic samples, etc. Among them, attentions have recently been paid to LOC techniques on bacteria (for example, E. coli) and animal cells over the DNA and protein levels. In particular, there has been a tremendously increasing demand for studies on a cell chip for handling and culturing an animal cell.

As the alternative studies on a cell chip, various studies have been reported, including an attempt to collect and absorb a single cell or a cell group within certain areas or their compartments, and a study of applying limited external stimuli to a small amount of cells while transferring the cells to certain sites.

However, among the studies on a cell chip, the animal cell culture in a portable micro cell incubator is mainly restricted by surroundings. In particular, although the most important requirement in the animal cell culture is a technique for supplying a gas and controlling temperature, the miniature cell culture system has a problem that it is impossible to supply gases such as oxygen and carbon dioxide and control a temperature to a suitable temperature level.

Therefore, the miniature animal cell incubator for culturing an animal cell needs a means for supplying oxygen and carbon dioxide, and a means for controlling a temperature of a culture medium to a suitable temperature level, the oxygen being necessarily required for basic metabolism of cells and the carbon dioxide being required for the control of pH in the culture medium.

Also, there has been required a means for self-supplying oxygen and carbon dioxide to a closed space such as portable devices rather than an open space.

### DISCLOSURE OF INVENTION

### Technical Problem

The present invention is designed to solve the problems of the prior art, and therefore it is an object of the present invention to provide a micro-scaled animal cell incubator capable of self-supplying gases required for the culture of animal cells and controlling temperature of a culture medium.

Also, it is another object of the present invention to provide a method for producing a micro-scaled animal cell incubator.

### Technical Solution

According to an aspect of the present invention, there is provided a micro-scaled animal cell incubator including an upper substrate having fine hot wires formed in a lower surface thereof and including a culture medium storage space; a lower substrate having a liquid and solid storage space and a gas flow channel formed in an upper surface thereof, the gas flow channel functioning to allow gases generated in the liquid and solid storage space to flow therethrough and the liquid and solid storage space being provided with fine hot wires; and a thin film interlayer formed between the culture medium storage space and the gas flow channel to allow generated gases to flow in the culture medium storage space.

In this case, the liquid and solid storage space may include two or more storage spaces, and one gas flow channel may be formed in each of the storage spaces. In particular, the liquid and solid storage space may include a storage space for storing H₂ O₂ and a storage space for storing NaHCO₃.

In addition, the culture medium storage space may have a plurality of the storage spaces coupled in series through the culture medium flow channel, or the culture medium storage space may be present in a plural number and have a plurality of the serial storage spaces coupled in series.

Furthermore, the gas flow channel may be formed with a compact structure in a position corresponding to each of a plurality of the storage spaces.

According to another aspect of the present invention, there is provided a micro-scaled animal cell incubator including a lower glass substrate having fine hot wires processed with a metal and formed in an upper surface thereof; a first PDMS film attached onto the lower glass substrate to form two or more liquid and solid storage spaces in a position corresponding to the fine hot wires of the lower glass substrate and gas flow channels coupled respectively to the liquid and solid storage spaces to allow generated gases to flow therethrough; a gas-permeable PDMS thin film attached onto the gas flow channels of the PDMS film to pass the generated gases therethrough; a second PDMS film attached onto the PDMS thin film and having a culture medium storage space for storing a culture medium; and an upper glass substrate attached onto the second PDMS film and having fine hot wires formed in a lower surface thereof, the fine hot wires being covered by the PDMS film.

According to still another aspect of the present invention, there is provided a method for producing a micro-scaled animal cell incubator, the method including: depositing a metal on a lower substrate; forming fine hot wire on the lower substrate by processing the deposited metal; attaching a polydimethylsiloxane (PDMS) film onto the lower substrate to form storage spaces for respectively storing an H₂O₂ solution and NaHCO₃ and gas flow channels formed in the respective storage spaces; attaching a gas-permeable PDMS thin film onto a PDMS film in which the gas flow channel is formed; attaching the PDMS film, in which the culture medium flow channel and the culture medium storage spaces are formed, onto the PDMS thin film; and attaching an upper substrate 100 onto the PDMS film in which the culture medium flow channel and the culture medium storage spaces are formed, the upper substrate 100 having fine hot wires 110 attached to the bottom thereof and the fine hot wires 110 being covered by the PDMS film.

### Advantageous Effects

As described above, the micro-scaled animal cell incubator according to the present invention may be useful to prevent the contamination in its closed space from external environments using a technology that is able to self-supply oxygen and carbon dioxide, the oxygen being required for the metabolism of animal cells and carbon dioxide being required for the pH control of culture medium.

Also, the micro-scaled animal cell incubator according to the present invention may be useful to maintain a constant temperature of the culture medium using its own temperature controller, and also to provide the most suitable culture environment to the micro-scaled incubator through the controlled supply of the oxygen and carbon dioxide.

And, the micro-scaled animal cell incubator according to the present invention may be useful to provide various suitable environments to various kinds of cells by widely varying a culture space of the cells.

### BRIEF DESCRIPTION OF THE DRAWINGS

FIG. 1 is a diagram illustrating a configuration of a micro-scaled animal cell incubator according to one exemplary embodiment of the present invention.

FIG. 2 is a diagram illustrating operations of producing a micro-scaled animal cell incubator according to one exemplary embodiment of the present invention.

FIG. 3 is a diagram illustrating a configuration of a gas flow channel in the micro-scaled animal cell incubator according to one exemplary embodiment of the present invention.

FIG. 4 is a diagram illustrating configurations of a culture medium flow channel/storage space in the micro-scaled animal cell incubator according to one exemplary embodiment of the present invention.

### BEST MODE FOR CARRYING OUT THE INVENTION

Hereinafter, exemplary embodiments of the present invention will now be described in detail with reference to the accompanying drawings, for the purpose of better understanding of the present invention as apparent to those skilled in the art. For the detailed description of the present invention, it is however considered that descriptions of known functions and their related configurations according to the exemplary embodiments of the present invention may be omitted when they are judged to make the gist of the present invention unclear.

Also, it is considered that parts that have the similar or substantially identical functions and effects throughout the accompanying drawings have the same reference numerals.

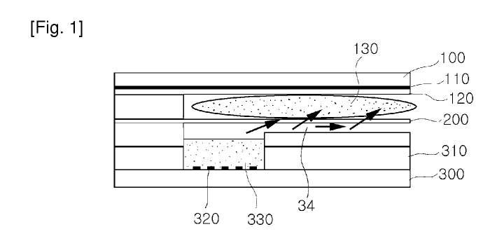

FIG. 1 is a diagram illustrating a configuration of a micro-scaled animal cell incubator according to one exemplary embodiment of the present invention.

The micro-scaled animal cell incubator according to the present invention includes an upper glass substrate 100 and a lower glass substrate 300. In this case, a culture medium flow channel/storage space 130 is formed in a lower surface of the upper glass substrate 100, and a liquid and solid storage space 330 for generating oxygen and carbon dioxide and a gas flow channel 340 are formed in an upper surface of the lower glass substrate 300 using polydimethylsiloxane (PDMS). And, a hot wire 110 for controlling a temperature of a culture medium is provided in a lower surface of the upper glass substrate 100, and a fine hot wire 320 for pyrolysis is provided in the liquid and solid storage space 330 disposed in the upper surface of the lower glass substrate 300. Also, a gas-permeable PDMS thin film 200 is formed between the gas flow channel 340 and the culture medium flow channel/storage space 130 to allow a gas such as oxygen or carbon dioxide generated in the liquid and solid storage space 330 to flow to the culture medium storage space through the gas flow channel 340.

The fine hot wire 320 made of a metal such as gold (Au) and chromium (Cr) is formed in the upper surface of the lower glass substrate 300 using a micro-processing technology such as an etching process. Here, the fine hot wire 320 is disposed in the liquid and solid storage space 330 to generate oxygen (O₂) and carbon dioxide (CO₂) by heating the storage space 330 in which NaHCO₃ and H₂O₂ are stored.

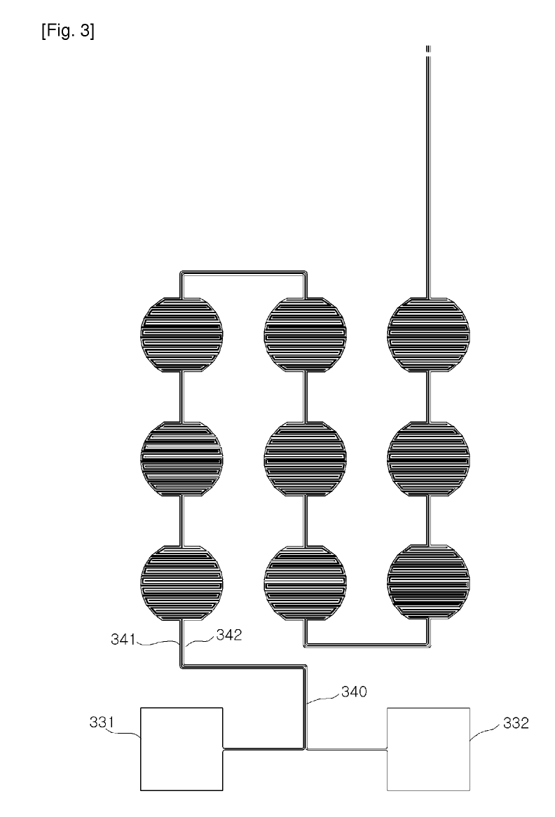

The liquid and solid storage space 330 and the gas flow channel 340 are formed in the upper surface of the lower glass substrate 300. The liquid and solid storage space 330 includes two or more storage spaces as show in FIG. 3, and one gas flow channel 340 is formed in each of the liquid and solid storage spaces 330. And, the respective gas flow channels 340 are formed adjacent to each other. Detailed description of the gas flow channel 340 will be described with reference to FIG. 3.

The gas-permeable PDMS thin film 200 is formed on a polydimethylsiloxane (PDMS) film that is attached to the lower glass substrate 300 to form the liquid and solid storage space 330 and the gas flow channel 340. The PDMS thin film 200 may be formed throughout the animal cell incubator, and also be formed in a restricted space between the culture medium storage space 130 and the gas flow channel 340. Therefore, gases generated in the liquid and solid storage space 330 permeate into a culture medium through the PDMS thin film 200 while moving through the gas flow channel 340.

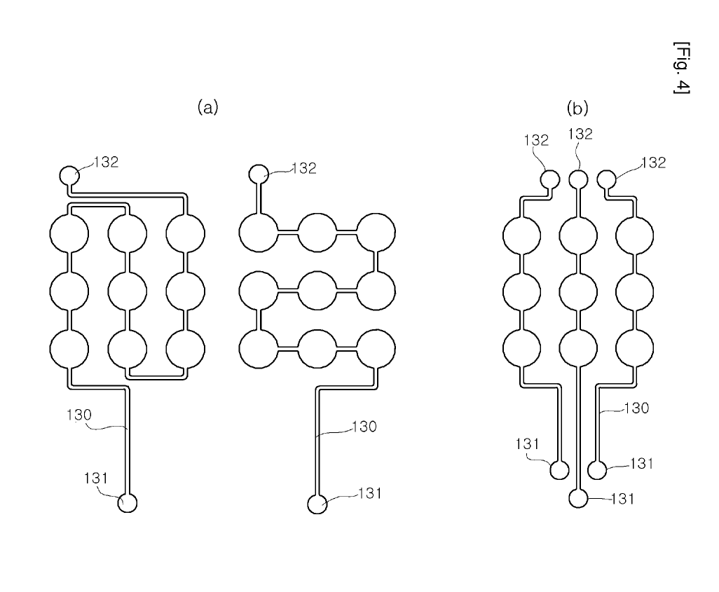

The culture medium storage space 130 is formed on the PDMS thin film 200. The culture medium storage space 130 is disposed between a polydimethylsiloxane (PDMS) thick film 120 and the PDMS thin film 200, both of which are formed of polydimethylsiloxane (PDMS), the PDMS thick film 120 being disposed in the lower surface of the upper glass substrate 100. The culture medium storage space 130 may be configured as shown in FIG. 4.

Also, the fine hot wires 110 are provided in the lower surface of the upper glass substrate 100. This is done to control a temperature of a culture medium. However, the fine hot wires 110 is covered by the PDMS thick film 120 to prevent electrolysis of the culture medium by electric current and remove an effect on animal cells, which prevents the direct contact with the culture medium.

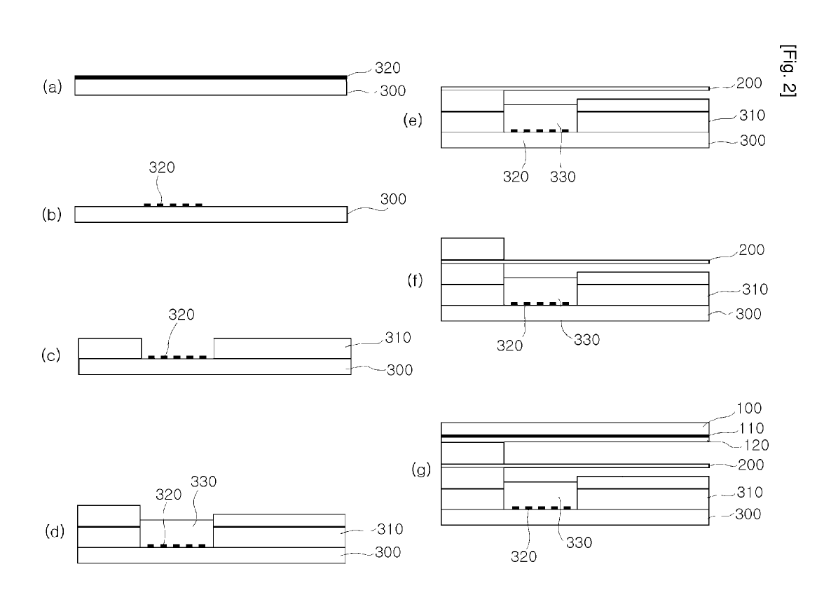

FIG. 2 is a diagram illustrating an operation of producing a micro-scaled animal cell incubator according to the present invention.

The production operation of the micro-scaled animal cell incubator proceeds in a direction from the lower glass substrate 300 to the upper glass substrate 100.

FIG. 2a shows an operation of depositing a metal on the lower glass substrate 300 so as to form fine hot wires on the lower glass substrate 300. First, Cr and Au are sequentially deposited on an upper surface of the lower glass substrate 300. The Cr and Au are preferably deposited with thicknesses of 0.01 and 0.03 μm, respectively, but the present invention is not particularly limited thereto.

FIG. 2b shows an operation of forming fine hot wires 320 by processing the metal deposited on the lower glass substrate 300. The fine hot wires 320 are formed by sequentially spin-coating HMDS and AZ 5214 photoresist on the glass substrate deposited with the metal, exposing the glass substrate to ultraviolet rays using a hot wire-patterned mask, sequentially etching Cr and Au except for the fine hot wires 320 and removing the photoresist with acetone. Here, the fine hot wires are preferably formed with a line width of 30 μm in a 5 mm*5 mm area. However, the present invention is not particularly limited thereto.

FIG. 2c shows an operation of attaching a PDMS thick film 310 onto the lower glass substrate 300 to form two storage spaces for storing an H₂O₂ solution NaHCO₃, respectively. In the present invention the storage spaces for storing an H₂O₂ solution NaHCO₃ are identical to the liquid and solid storage space. The liquid and solid storage space is divided into two storage spaces: an H₂O₂ solution is stored in one storage space to generate oxygen, and NaHCO₃ is stored in the other storage space to generate carbon dioxide.

FIG. 2d shows an operation of injecting H₂O₂ and NaHCO₃ into the liquid and solid storage space and attaching another PDMS film having a gas flow channel formed therein. Here, a gas flow channels 14 and a liquid and solid storage space are formed on the attached PDMS film, as shown in FIG. 3. Therefore, when the PDMS film having the gas flow channel 14 formed therein is attached to PDMS thick film 310, the gas flow channel are formed while being coupled to the liquid and solid storage space.

FIG. 2e is an operation of attaching a gas-permeable PDMS thin film onto the PDMS film having a gas flow channel 14 formed therein. Here, the gas-permeable PDMS thin film is preferably formed with a thickness of 250 μm, but the present invention is not particularly limited thereto.

FIG. 2f is an operation of attaching a PDMS film having a culture medium flow channel and a culture medium storage space formed therein to the gas-permeable PDMS thin film. Herein, the culture medium flow channel and the culture medium storage space formed in the PDMS film are identical to those as shown in FIG. 4.

FIG. 2g is an operation of attaching an upper glass substrate 100 onto the PDMS film having a culture medium flow channel and a culture medium storage space formed therein, wherein the upper glass substrate 100 has fine hot wires 110 attached to a lower portion thereof, and the fine hot wires 110 are covered by the PDMS film.

The micro-scaled animal cell incubator is produced through these operations.

FIG. 3 is a diagram illustrating a configuration of a gas flow channel in the micro-scaled animal cell incubator according to the present invention.

The liquid and solid storage space 330 is composed of two storage spaces 331 and 332 in which H₂O₂ and NaHCO₃ are stored, respectively. For example, H₂O₂ is stored in one liquid and solid storage space 331 to generate oxygen by means of heat generated in the fine hot wires, and the generated oxygen moves through the gas flow channel 341. And, NaHCO₃ is stored in the other liquid and solid storage space 332 to generate carbon dioxide by means of heat generated in the fine hot wires, and the generated carbon dioxide moves through the gas flow channel 342.

The decomposition of H₂O₂ into water and oxygen in the liquid and solid storage space 331 is carried out according to the following chemical reaction.

2 H₂O₂(l)->2 H₂O (l)+O₂ (g)

The generated oxygen flows out from the H₂O₂ storage space 331 and moves along the gas flow channel 341 coupled to the H₂O₂ storage space 331. The gas flow channel 341 is formed with a very compact structure in a predetermined position, as shown in FIG. 3. The gas flow channel 341 is compactly formed in the compact structure by repeatedly arranging a gas flow channel in the opposite direction of the gas flow channel 341 (e.g., in a zigzag manner). However, this structure of the gas flow channel 341 is applicable as one exemplary embodiment of the present invention. However, the gas flow channel 341 is preferably compactly formed in a position corresponding to a storage space for storing a culture medium in the present invention since the gas flow channel 341 is formed for the purpose of supplying oxygen or carbon dioxide to a culture medium storage space through a PDMS thin film.

The oxygen generated in the liquid storage space 331 is supplied to the culture medium storage space while moving through the gas flow channel 341, and the remaining oxygen is discharged through an outlet. This is done to prevent an internal pressure of the gas flow channel 341 from increasing excessively.

The decomposition of NaHCO₃ into water and carbon dioxide in the other solid storage space 332 is carried out according to the following chemical reaction.

2 NaHCO₃ (s)->Na₂CO₃ (s)+H₂O (l)+CO₂ (g)

In the same manner as in the oxygen generated by the decomposition of H₂O₂, the generated carbon dioxide flows from the solid storage space 332 through the gas flow channel 342, and permeates through the PDMS thin film to supply the carbon dioxide to a culture medium, and the remaining carbon dioxide is discharged.

As shown in FIG. 3, the channels through which the oxygen and the carbon dioxide generated respectively in the liquid and solid storage spaces 331 and 332 flow are provided respectively in the liquid and solid storage spaces 331 and 332, and they are formed along the same path.

FIG. 4 is a diagram illustrating configurations of a culture medium flow channel/storage space in the micro-scaled animal cell incubator according to the present invention.

The configuration of the culture medium flow channel/storage space is formed on the PDMS thin film of the micro-scaled animal cell incubator. The configuration of the culture medium flow channel/storage space includes a culture sample inlet 131 for injecting a mixture of a liquid-phase culture sample and an animal cell to be cultured, a plurality of culture sample storage spaces 130 for storing the injected culture sample while allowing the culture sample to move therethrough, and a culture sample outlet 132 for discharging gases filled in micro spaces.

The inner part of the culture medium flow channel/storage space may be treated with collagen, fobronectin or poly-L-lysine to culture animal cells that grow while being adhered to the inner part of the culture medium flow channel/storage space.

The animal cells and the culture medium injected through the culture sample inlet 131 are sequentially filled in culture medium storage spaces while flowing through a micro channel, and the gases filled in the micro spaces are discharged through a culture sample outlet 132. Also, the culture sample outlet 132 is used to recovery the cultured cells and the culture medium after the use of the animal cell incubator.

After the injection of the animal cells and the culture medium is completed, the culture medium sample inlet 131 and the culture medium sample outlet 132 are sealed to cut off the culture medium sample inlet 131 and the culture medium sample outlet 132 from the external environment, and an electric current flows in the fine hot wires to initiate the pyrolysis of H₂O₂ and NaHCO₃. In this case, the amounts of the oxygen and carbon dioxide to be supplied may be adjusted by controlling the electric current applied to the fine hot wires.

Meanwhile, a temperature of the culture medium is maintained to a temperature level of 37? by applying an electric current to the fine hot wires 110 arranged in the upper glass substrate. This is one exemplary embodiment of the present invention, but the present invention is not particularly limited thereto.

The culture medium flow channel/storage space may be provided to culture the same cells under the same culture conditions by arranging storage spaces for cell culture in series, as shown in FIG. 4a. Also, the culture medium flow channel/storage space in which storage spaces are arranged in series may be provided in plural number, as shown in FIG. 4b. In this case, more than two kinds of animal cells may be cultured under the same culture conditions, or different animal cells may be cultured under the different culture conditions. Therefore, the configuration of the culture medium flow channel/storage space may be widely changed and modified according to the field of its applications.

While the present invention has been shown and described in connection with the exemplary embodiments, it will be apparent to those skilled in the art that modifications and variations can be made without departing from the spirit and scope of the invention as defined by the appended claims.

1. A micro-scaled animal cell incubator, comprising:
an upper substrate having fine hot wires formed in a lower surface thereof and including a culture medium storage space;
a lower substrate having a liquid and solid storage space and a gas flow channel formed in an upper surface thereof, the gas flow channel functioning to allow gases generated in the liquid and solid storage space to flow therethrough and the liquid and solid storage space being provided with fine hot wires; and
a thin film interlayer formed between the culture medium storage space and the gas flow channel to allow generated gases to flow in the culture medium storage space.

2. The micro-scaled animal cell incubator of claim 1, wherein the liquid and solid storage space includes two or more storage spaces, and one gas flow channel is formed in each of the storage spaces.

3. The micro-scaled animal cell incubator of claim 2, wherein the liquid and solid storage space includes a storage space for storing H₂O₂ and a storage space for storing NaHCO₃.

4. The micro-scaled animal cell incubator of claim 1, wherein the culture medium storage space has a plurality of storage spaces coupled through a culture medium flow channel.

5. The micro-scaled animal cell incubator of claim 4, wherein the culture medium storage space has a plurality of the storage spaces coupled in series through the culture medium flow channel.

6. The micro-scaled animal cell incubator of claim 4, wherein the culture medium storage space has a plurality of the structures with a plurality of storage spaces coupled in series.

7. The micro-scaled animal cell incubator of claim 6, wherein the gas flow channel is formed with a compact structure in a position corresponding to each of a plurality of the storage spaces.

8. A method for producing a micro-scaled animal cell incubator, the method comprising:
depositing a metal on a lower substrate;
forming fine hot wire on the lower substrate by processing the deposited metal;
attaching a polydimethylsiloxane (PDMS) film onto the lower substrate to form storage spaces for respectively storing an H₂O₂ solution and NaHCO₃ and gas flow channels formed in the respective storage spaces;
attaching a gas-permeable PDMS thin film onto a PDMS film in which the gas flow channel is formed;
attaching the PDMS film, in which the culture medium flow channel and the culture medium storage spaces are formed, onto the PDMS thin film; and
attaching an upper substrate 100 onto the PDMS film in which the culture medium flow channel and the culture medium storage spaces are formed, the upper substrate 100 having fine hot wires 110 attached to the bottom thereof and the fine hot wires 110 being covered by the PDMS film.

9. The method of claim 8, wherein the depositing of a metal on a lower substrate is carried out by sequentially depositing chromium (Cr) and gold (Au) on an upper surface of the lower substrate.

10. The method of claim 8, wherein the forming of fine hot wires comprises:
sequentially spin-coating hexamethyldisilazane (HMDS) and AZ 5214 photoresist on the lower substrate deposited with the metal;
exposing the spin-coated lower substrate to ultraviolet rays using a hot wire-patterned mask;
sequentially etching Cr and Au except for the fine hot wires 320; and
removing the photoresist with acetone.

11. The method of claim 8, wherein each of the upper substrate and the lower substrate is a glass substrate.

12. A micro-scaled animal cell incubator, comprising:
a lower glass substrate having fine hot wires that are processed with a metal and formed in an upper surface thereof;
a first PDMS film attached onto the lower glass substrate to form two or more liquid and solid storage spaces in a position corresponding to the fine hot wires of the lower glass substrate and gas flow channels coupled respectively to the liquid and solid storage spaces to allow generated gases to flow therethrough;
a gas-permeable PDMS thin film attached onto the gas flow channels of the PDMS film to pass the generated gases therethrough;
a second PDMS film attached onto the PDMS thin film and having a culture medium storage space for storing a culture medium; and
an upper glass substrate attached onto the second PDMS film and having fine hot wires formed in a lower surface thereof, the fine hot wires being covered by the PDMS film.

13. The micro-scaled animal cell incubator of claim 12, wherein the gas flow channel is compactly formed in a position corresponding to the culture medium storage space.

14. The micro-scaled animal cell incubator of claim 13, wherein the culture medium storage space includes a plurality of storage spaces.
